(19) United States
(12) Patent Application Publication
(10) Pub. No.: US 2018/0133171 A1
(43) Pub. Date: May 17, 2018

(54) COMPOSITION INCLUDING 3-BROMO-4,5-DIHYDROXYBENZALDEHYDE COMPOUND AS EFFECTIVE COMPONENT FOR PROTECTING AND TREATING SKIN CELL AGANIST ULTRAVIOLET

(71) Applicant: Jeju National University Industry-Academic Cooperation Foundation, Jeju-Si (KR)

(72) Inventors: Jin-Won Hyun, Jeju-si (KR); Hee-Kyoung Kang, Jeju-si (KR); Nam-Ho Lee, Jeju-si (KR); Sung-Wook Chae, Daejeon (KR)

(73) Assignee: Jeju National University Industry-Academic Cooperation Foundation, Jeju-Si (KR)

(21) Appl. No.: 15/861,440

(22) Filed: Jan. 3, 2018

Related U.S. Application Data
(62) Division of application No. 13/846,191, filed on Mar. 18, 2013.

(30) Foreign Application Priority Data
Jun. 28, 2012 (KR) 10-2012-0069710

(51) Int. Cl. A61K 31/11; A61K 8/34; A61Q 17/04

(52) U.S. Cl. CPC A61K 31/11; A61Q 17/04; A61K 8/347

(57) ABSTRACT

There is provided a composition including a 3-bromo-4,5-dihydroxybenzaldehyde (BDB) for protecting human skin keratinocyte from ultraviolet, in which the BDB has a photo-protective effect against cell damage caused by ultraviolet in human HaCaT skin keratinocyte, activity of removing a free radical, and ultraviolet absorption activity, and inhibits formations of lipid peroxidation and protein carbonyl, inhibits DNA damage, protects a cell, and thereby exhibits cell protective activity. Thus, the BDB decreases apoptosis induced by ultraviolet, and then protect a cell to recover cell viability.

### CROSS-REFERENCE TO RELATED APPLICATIONS

This application claims the priority of Korean Patent Application No. 2012-69710 filed on Jun. 28, 2012, in the Korean Intellectual Property Office, the disclosure of which is incorporated herein by reference.

### BACKGROUND OF THE INVENTION

### Field of the Invention

The present invention relates to an ultraviolet absorption composition capable of inhibiting skin damage caused by ultraviolet and protecting skin from ultraviolet.

### Description of the Related Art

In general, when ultraviolet included in sunlight is excessively and directly exposed to skin, a formation of red spots or a production of melanin in skin cells may be promoted to cause a generation of freckles or blemishes spots. Further, sebum secreted in epidermis is reacted to produce lipid peroxide, and thereby skin problems may be caused. Furthermore, in severe cases, skin cancer may be caused. Ultraviolet rays are classified into UV-A (320 nm to 400 nm), UV-B (280 nm to 320 nm) and UV-C (200 nm to 280 nm) according to a wavelength, and among them, it has been known that ultraviolet that reach the top of the ground and then affect a human body are UV-A and UV-B.

It has been known that an UVB exposure allows free radicals to be greatly produced and reactive oxygen species (ROS) to be greatly generated in skin, induces oxidative stress to cell components, such as DNA, cell membrane, and protein, and thus ages the skin. For this reason, various studies on natural antioxidants for protecting skin damage induced by UVB are currently underway.

Meanwhile, a 3-Bromo-4,5-dihydroxybenzaldehyde (BDB) can be isolated from red alga such as Rhodomela confervoides, Polysiphonia morrowii, and Polysiphonia urceolata. The BDB has an antiviral effect to hematopoietic necrosis virus and infectious pancreatic necrosis virus, and also a 1,1-diphenyl-2-picrylhydrazyl radical removal effect. However, an effect of the BDB in protecting against UVB is not known.

### CITATION LIST

### Patent Document

Patent Document 1: Korean Patent Publication No. 2002-0042020

### SUMMARY OF THE INVENTION

Accordingly, the inventors of the present invention found that a BDB has an anti-oxidative effect according to free radical removal activity in a cell, in which the free radical is generated due to an irradiation of ultraviolet, an effect in absorbing such ultraviolet itself, and also a cell protective effect according to an inhibition of apoptosis induced by an irradiation of ultraviolet. Thus, the inventors completed the present invention.

Accordingly, an object of the present invention is to provide a pharmaceutical composition for inhibiting and treating skin damage caused by ultraviolet, in which the composition includes a 3-bromo-4,5-dihydroxybenzaldehyde (BDB) or salt thereof as an effective component.

In addition, another object of the present invention is to provide an ultraviolet absorption composition including a 3-bromo-4,5-dihydroxybenzaldehyde or salt thereof as an effective component.

Still another object of the present invention is to provide a cosmetic composition for inhibiting skin damage caused by ultraviolet and protecting skin from ultraviolet, in which the composition includes a 3-bromo-4,5-dihydroxybenzaldehyde (BDB) or salt thereof as an effective component.

In order to achieve the objects of the present invention as described above, according to an aspect of the present invention, there is provided a pharmaceutical composition for inhibiting and treating skin damage caused by ultraviolet, in which the composition includes a 3-bromo-4,5-dihydroxybenzaldehyde (BDB) or salt thereof as an effective component.

According to an example of the present invention, the 3-bromo-4,5-dihydroxybenzaldehyde may have activity of removing intracellular free radicals generated by an ultraviolet absorption and ultraviolet irradiation or activity of inhibiting apoptosis induced by ultraviolet in a cell.

According to an example of the present invention, the pharmaceutical composition may be a composition in a type for external application of skin selected from the group consisting of cream, gel, a patch, a spraying agent, ointment, a hardening agent, lotions, liniments, pastes, and cataplasma.

According to an example of the present invention, the composition may include a BDB in a concentration of 10 μM to 40 μM.

In addition, the present invention provides an ultraviolet absorption composition including a 3-bromo-4,5-dihydroxybenzaldehyde or salt thereof as an effective component.

Furthermore, the present invention provides a cosmetic composition for inhibiting skin damage caused by ultraviolet and protecting skin from ultraviolet, in which the composition includes a 3-bromo-4,5-dihydroxybenzaldehyde or salt thereof as an effective component.

According to an example of the present invention, the cosmetic composition may be formulated into skin lotions, skin softeners, skin toners, astringents, lotions, milky lotions, moisture lotions, nutrition lotions, massage creams, nutrition creams, moisture creams, hand creams, essences, nutrition essences, packs, soaps, shampoos, cleansing foams, cleansing lotions, cleansing creams, body lotions, body cleansers, milky liquids, lipsticks, make-up bases, foundations, press powders, loose powders, or eye shadows.

According to an example of the present invention, the composition may include a BDB in a concentration of 10 μM to 40 μM.

### BRIEF DESCRIPTION OF THE DRAWINGS

The above and other aspects, features and other advantages of the present invention will be more clearly understood from the following detailed description taken in conjunction with the accompanying drawings, in which:

FIG. 1a is a graph illustrating cell viabilities confirmed through a MTT assay when BDBs with various concentrations were treated. Here, * represents data which is significantly different from a control group, and ** represents data which is significantly different from the cell treated with UVB (p<0.05).

FIG. 1b is a graph illustrating the results of analyzing the cell viability by using a MTT assay when UVB was irradiated after treating BDBs with various concentrations to a cell. Here, represents data which is significantly different from a control group, and ** represents data which is significantly different from the cell treated with UVB (p<0.05).

FIG. 1c is a graph illustrating the results of analyzing the level of DPPH radical by using a spectrophotometer and the results of analyzing the intracellular ROS level generated by H₂O₂ or UVB by using a spectrofluorometer.

FIG. 1d is diagrams illustrating, in a peak, the results of analyzing DMPO/OOH by-products and a superoxide anion produced through a reaction of xanthine and xanthine oxidase with DMPO by using an ESR spectrometry.

FIG. 1e is diagrams illustrating, in a peak, the results of analyzing DMPO/.OH by-products that is a product produced by reacting hydroxyl radical produced by a Fenton reaction (H₂O₂+FeSO₄) with DMPO by using an ESR spectrometry.

FIG. 2 is a graph illustrating UVB absorption ability of a BDB that was analyzed at 200 nm to 500 nm by using an ultraviolet/visible ray spectroscopic measurement. Peaks 1 and 2 represent the positions at 288 nm and 357 nm, respectively.

FIG. 3a is a graph illustrating the results of analyzing lipid peroxidation by measuring the level of 8-isoprostane for analyzing an effect on oxidative stress in a cell when a BDB was treated.

FIG. 3b is photographs illustrating the result of observing by using a fluorescence microscope after DPPP fluorescence staining in order to analyze an effect on oxidative stress in a cell when a BDB was treated.

FIG. 3c is a graph illustrating the results of analyzing protein oxidation by measuring the amount of carbonyl formation in order to analyze an effect on oxidative stress in a cell when a BDB was treated. Here, * represents data which is significantly different from a control group (p<0.05), and ** represents data which is significantly different from the cell irradiated with UVB (p<0.05).

FIG. 3d is photographs and a graph illustrating images and ratio of DNA damage in a cell by performing a comet assay. Here, * represents data which is significantly different from a control group (p<0.05), and ** represents data which is significantly different from the cell irradiated with UVB (p<0.05).

FIG. 4a is photographs and a graph illustrating the results of observing and quantizing apoptotic body (arrows) through a fluorescence microscope in a cell stained with a Hoechst 33342 dye. Here, * represents data which is significantly different from a control group (p<0.05), and ** represents data which is significantly different from the cell irradiated with UVB (p<0.05).

FIG. 4b is graphs illustrating the results of analyzing an apoptotic sub-G₁DNA content by using a flow cytometry after staining with propidium iodide.

FIG. 4c is a graph illustrating the results of quantizing a DNA fragmentation bonded with histone of cytoplasm by using a kit. Here, * represents data which is significantly different from a control group (p<0.05), and ** represents data which is significantly different from the cell irradiated with UVB (p<0.05).

### DETAILED DESCRIPTION OF THE PREFERRED EMBODIMENT

Exemplary embodiments of the present invention will now be described in detail with reference to the accompanying drawings.

The present invention was completed by confirming that a 3-bromo-4,5-dihydroxybenzaldehyde (BDB) had an effect in inhibiting oxidative stress of cell by inhibiting a production of reactive oxygen species caused by ultraviolet B (UVB), so that the BDB improves cell viability of cell (human epidermal keratinocyte), ultimately inhibits apoptosis caused by ultraviolet rays, and protects cells.

It has been known that UVB induces oxidative stress by producing reactive oxygen species, and thus induces damages of various skin organization cells. For this reason, a photo-aging process is ultimately accelerated. According to the present invention, it was found that the BDB of the present invention is suitable as the composition having an antioxidant effect through comparison experiments of free radical removal ability, UVB absorption ability, ability of inhibiting cytotoxicity and oxidative death caused by UVB irradiation, and an intracellular antioxidant effect.

More specifically, according to an example of the present invention, it was confirmed the fact that the BDB of the present invention inhibits apoptosis caused by UVB, and thus increases cell viability depending on its concentrations.

UVB light accelerates ROS generation and induces oxidative stress. A treatment of BDB is effective in inhibiting oxidative stress induced by UVB radiation in skin keratinocyte. When the BDB is treated, the BDB removes ROS in the cell exposed to UVB irradiation, so that a production of ROS is reduced. According to an example of the present invention, it was confirmed that the BDB removes intracellular DPPH radicals, superoxide anions, hydroxyl radicals, and ROS.

A cell protective effect of a BDB is relevant to UV absorption ability as illustrated in an absorption spectrum of the BDB. Therefore, the BDB can decrease the number of photons that attack a cell. Among many light protectors, natural antioxidants can effectively decrease oxidative skin damage caused by UVB. Accordingly, according to the present invention, it can be confirmed that the BDB can protect skin cells by directly absorbing UVB.

A BDB is a phenol-based compound and has an antioxidant effect by removing ROS. Cell damage caused by UVB is multifaceted. For example, cell membrane lipids are prone to be damaged by UVB. A BDB protects cell membrane lipid from UVB. In addition, UVB radiation induces fragmentation of DNA strand. According to an example of the present invention, the BDB exhibits small DNA tail confirmed by a comet assay. Accordingly, it may be inferred that the BDB of the present invention inhibits lipid peroxidation to protect a cell from UVB.

Protein carbonylation functions as a biomarker in the protein damage induced by oxidative stress. Further, when modified protein carbonyl groups are accumulated, a cell function is inhibited. According to an example of the present invention, the BDB reduces the level of carbonylated protein generated by UVB.

In addition, according to an example of the present invention, it can be confirmed that the BDB is capable of protecting DNA in a cell from damage caused by UVB.

UVB radiation is a strong inducing agent of apoptosis, and produces ROS. From the following Example 8, it can be confirmed that cell death caused by apoptosis generated by UVB radiation is inhibited by decreasing the number of apoptotic body and DNS fragmentation when a BDB is treated.

In other words, the BDB having the aforementioned properties may be useful to inhibit skin damage caused by ultraviolet and protect skin from ultraviolet, so that the BDB may be used for a pharmaceutical composition for inhibiting and treating skin damage caused by ultraviolet, in which the pharmaceutical composition includes the BDB as an effective component. Further, the BDB compound itself has an ultraviolet absorption effect, so that the BDB can be used for a composition as an ultraviolet absorption composition including the BDB as an effective component.

Further, the 3-bromo-4,5-dihydroxybenzaldehyde (BDB) included in the composition according to the present invention as an effective component may be used in a type of salts, preferably pharmaceutically acceptable salts. Such salts may be preferably acid addition salts produced by pharmaceutically acceptable free acid, and examples of such a free acid may include an organic acid and inorganic acid. Examples of such an organic acid may include, but are not limited to, citric acid, acetic acid, lactic acid, tartaric acid, maleic acid, fumaric acid, formic acid, propionic acid, oxalic acid, trifluoroacetic acid, benzoic acid, gluconic acid, methasulfonic acid, glycolic acid, succinic acid, 4-toluenesulfonic acid, glutamic acid, and aspartic acid. Furthermore, examples of such an inorganic acid may include, but are not limited to, hydrochloric acid, bromic acid, sulfuric acid, and phosphoric acid.

The 3-bromo-4,5-dihydroxybenzaldehyde (BDB) compound according to the present invention may be naturally isolated, or may be produced by using a chemical synthetic method that is known in the prior art.

The composition according to the present invention including the BDB as an effective component may be a pharmaceutical composition.

The pharmaceutical composition according to the present invention may be prepared by using adjurvants that are pharmaceutically suitable and physiologically acceptable in addition to such an effective component. Examples of such adjurvants may include excipient, a disintegrating agent, a sweeting agent, a bonding agent, a coating agent, a blowing agent, a lubricant, a modifier, a flavouring agent, or the like.

The pharmaceutical composition may preferably be formulated by further including at least one pharmaceutically acceptable carrier in addition to the aforementioned effective component in order for an administration.

A formulation type of the pharmaceutical composition may be granules, powders, tablets, covered tablets, capsules, suppository, liquid formulations, syrups, juices, suspensions, an emulsion, medicinal drops, injectable liquid formulations, or the like. For example, in order to formulate in a type of tablets or capsules, an effective component may be bonded with an oral, nontoxic, pharmaceutically acceptable inert carrier, such as ethanol, glycerol, water, or the like. Further, in the case of need or necessary, a suitable bonding agent, lubricant, disintegrating agent and a color former may be included in a mixture. Examples of suitable bonding agent may include, but are not limited to, natural sugars, such as starch, gelatin, glucose, or beta-lactose, natural and synthetic gums, such as corn sweeting agents, acacia, tragacanth, or sodium oleate, sodium stearate, magnesium stearate, sodium benzoate, sodium acetate, sodium chloride, and the like. Examples of the disintegrating agent may include, but are not limited to, starch, methyl cellulose, agar, bentonite, xanthane gum, and the like. In the composition formulated in a liquid solution, as a pharmaceutically acceptable carrier and a suitable material for sterilization and human body, saline solution, sterile water, Ringer's solution, buffered saline, albumin injection solution, dextrose solution, maltodextrin solution, glycerol, ethanol, and a mixture of at least one therefrom may be used, and if necessary, other general additives, such as antioxidants, a buffer solution, or bacteristat may be included. Furthermore, it may be formulated in tables, granules, capsules, pills, or injectable tablets such as aqueous solution, suspensions, and emulsions by additionally adding diluent, a dispersing agent, surfactant, a bonding agent, and a lubricant. Further, it may preferably be formulated according to all the diseases or components by using the method as disclosed in Remington's Pharmaceutical Science, Mack Publishing Company, Easton Pa. as a proper method in the prior art.

According to an example of the present invention, a pharmaceutically effective amount of the BDB according to the present invention may be 10 μM to 40 μM and preferably 30 μM. However, the pharmaceutically effective amount may be properly changed according to a degree of skin damage, an age, a body weight, a health condition, sex, an administration route, a treatment period of a patient, and the like.

According to an example of the present invention, the pharmaceutical composition of the present invention may be a skin composition for external use. Further, the composition of the present invention has an ultraviolet absorption effect, so that it may be used as a skin composition for external use to be applied to skin, or sunscreen as a cosmetic composition for absorbing ultraviolet, and the like.

The pharmaceutically skin composition for external use according to the present invention may be prepared and used in a type of a pharmaceutically skin composition for external use, such as cream, gel, a patch, a spraying agent, ointment, a hardening agent, lotions, liniments, pastes, and cataplasma as a skin composition for external use having a skin protective effect from ultraviolet. However, the present invention is not limited thereto.

Further, the composition of the present invention may be a cosmetic composition for inhibiting skin damage caused by ultraviolet and protecting skin from ultraviolet, in which the composition includes a BDB as an effective component.

In a case in which the composition of the present invention is prepared in a cosmetic composition, the composition of the present invention may include components that are generally used for the cosmetic composition as well as the BDB as disclosed above, and for example, general adjurvants such as antioxidant, stabilizer, a solubilizing agent, vitamins, pigments, and flavouring, and a carrier.

Further, the composition of the present invention may be used by mixing organic sunscreen agents that have conventionally been used within the range, in which a skin protective effect is not damaged by reacting with a BDB, in addition to the BDB as disclosed above.

Examples of such organic sunscreen agents may include at least one selected from the group consisting of glyceryl PABA, drometrizole trisiloxane, drometrizole, digalotrioleate, disodiumphenylbenzimidazoletetrasulfonate, diethylhexylbutamidotriazone, diethylaminohydroxybenzoylhexylbenzoate, DEA-methoxycinnamate, a mixture of Lowsone and dihydroxyacetone, methylene bis-benzotriazolyl tetramethylbutylphenol, 4-methylbenzylidene camphor, menthyl anthranilate, benzophenone-3(oxybenzone), benzophenone-4, benzophenone-8(dioxypebenzone), butylmethoxydibenzoylmethane, bisethylhexyloxyphenolmethoxyphenyltriazine, cinoxate, ethyldihydroxypropyl PABA, octocrylate, ethylhexyldimethyl PABA, ethylhexylmethoxycinnamate, ethylhexyl salicylate, ethylhexyl triazone, isoamyl-p-methoxycinnamate, polysilicone-15 (dimethicodiethylbenzalmalonate), terephthalylidene dicamphor sulfonic acid, salts thereof, TEA-salicylate, and aminobenzoic acid (PABA).

Products that can use the cosmetic composition of the present invention may include cosmetic products, such as an astringent, skin lotion, nutrition lotion, all kinds of creams, essences, packs, and foundations, cleansing, face cleansing products, soaps, treatments, cosmetic solutions, and the like.

A specific formulation of the cosmetic composition according to the present invention includes skin lotions, skin softeners, skin toners, astringent, lotions, milk lotions, moisture lotions, nutrition lotions, massage creams, nutrition creams, moisture creams, hand creams, essences, nutrition essences, packs, soaps, shampoos, cleansing foams, cleansing lotions, cleansing creams, body lotions, body cleansers, an emulsion, lipsticks, make-up bases, foundations, press powders, loose powders, eye shadows, and the like.

According to a preferable embodiment of the present invention, a content of the BDB of the present invention is 10 μM to 40 μM and preferably 30 μM relative to the total weight of the composition. When the content of the BDB is less than 10 μM, an ultraviolet absorption effect may be greatly decreased. On the other hand, when it exceeds 40 μM, skin irritation may be caused, and also a dosage form problem may be caused.

Meanwhile, the cosmetic composition according to the present invention may be formulated by including the BDB inside nano-liposome, and then stabilizing the BDB. When the compound is included inside the nano-liposome, the compound is stabilized, so that problems such as precipitation, discolorization, and a smell change may be solved, and a percutaneous absorption rate and solubility of the component may be increased when formulating into a dosage form. Therefore, effectiveness to be expected from the compound may be maximally exhibited.

The nano-liposome used in the present invention means liposome having an average particle diameter of 10 to 500 nm with a type of a general liposome. According to a preferable embodiment of the present invention, an average particle diameter of the nano-liposome is 50 to 300 nm. When the average particle diameter of the nano-liposome exceeds 300 nm, among the technical effects to be achieved in the present invention, an improvement of dermal penetration and an improvement of dosage form stability may be very weak. The nano-liposome used for stabilizing the BDB compound according to the present invention may be prepared by a mixture including polyol, an oil component, surfactant, phospholipid, fatty acid, and water.

The polyol used in the nano-liposome of the present invention includes, but is not limited to, preferably, at least one selected from the group consisting of propylene glycol, dipropylene glycol, 1,3-butylene glycol, glycerin, methyl propanediol, isopropylene glycol, pentylene glycol, erythritol, xylitol, sorbitol, and mixtures thereof. The used amount thereof is 10 to 80 wt % and preferably 30 to 70 wt % relative to the total weight of the nano-liposome.

The oil component used for preparing the nano-liposome of the present invention may include various oils that are known in the prior art, but preferably hydrocarbon-based oils such as hexadecane and paraffin oils, silicone oils such as ester-based synthetic oil, dimethicone and cycliomethicone-based oils, animal and vegetable oils such as sunflower oil, corn oil, soybean oil, avocado oil, sesame seed oil, and fish oil, sphingoid liqid such as ethoxylated alkylether-based oil, propoxylated alkyleter-based oil, phytosphingosine, sphingosine, and sphinganine, cerebroside cholesterol, cytosterol cholesteryl sulfate, cytosteryl sulfate, C₁₀ to C₄₀ fatty alcohol and mixtures thereof. The used amount thereof may be 1.0 to 30.0 wt % and preferably 3.0 to 20.0 wt % relative to the total weight of the nano-liposome.

The surfactant used for preparing the nano-liposome of the present invention may include any things that are known in the prior art. Examples thereof may include anionic surfactant, cationic surfactant, ampholytic surfactant, and nonionic surfactant. Preferably, anionic surfactant and nonionic surfactant may be used. Specific examples of the anionic surfactant may include alkylacylglutamate, alkyl phosphate, alkyl lactylate, dialkyl phosphate, and trialkyl phosphate. Specific examples of the nonionic surfactant may include alkoxylated alkyl ether, alkoxylated alkyl ester, alkylpolyglycoside, polyglyceryl ester, and sugar ester. Still most preferable examples of the surfactant may include polysorbates belonging to nonionic surfactant. The used amount thereof may be 0.1 to 10 wt % and preferably 0.5 to 5.0 wt % relative to the total weight of the nano-liposome.

The phospholipid that is another component used for preparing the nano-liposome of the present invention may be ampiphilic lipid, and examples thereof may include natural phospholipid (for example, egg yolk lecithin or soybean lecithin, sphingo myelin) and synthetic phospholipid (for example, dipalmitoyl phosphatidylcholine or hydrogenated lecithin), and preferably lecithin. Especially, unsaturated lecithin or saturated lecithin that is naturally derived and extracted from a soybean or the yolk of an egg is preferable. In general, in natural lecithin, the amount of phosphatidylcholine is 23 to 95% and the amount of phosphatidylethanolamine is 20% or less. In a preparation of the nano-liposome of the present invention, the used amount of the phospholipid is 0.5 to 20.0 wt % and preferably 2.0 to 8.0 wt % relative to the total weight of the nano-liposome.

The fatty acid used for preparing the nano-liposome of the present invention is higher fatty acid, and preferably includes saturated or unsaturated fatty acid of C₁₂ to C₂₂ alkyl chains, such as lauric acid, myristic acid, palmitic acid, stearic acid, oleic acid, and linoleic acid. The used amount thereof may be 0.05 to 3.0 wt % and preferably 0.1 to 1.0 wt % relative to the total weight of the nano-liposome.

The water used for preparing the nano-liposome of the present invention may generally be deionized distilled water, and the used amount thereof may be 5.0 to 40 wt % relative to the total weight of the nano-liposome.

The preparation of the nano-liposome may be achieved through various methods that are known in the prior art, and most preferably, the nano-liposome may be prepared by applying the mixture including the aforementioned components to a high pressure homogenizer. The preparation of the nano-liposome using the high pressure homogenizer may be performed under various conditions (for example, a pressure, the number of performances, and the like) according to the desired particle size, and preferably the nano-liposome may be prepared by passing the mixture through the high pressure homogenizer one to five times under pressure of 600 to 1200 bar.

The cosmetic composition for protecting skin according to the present invention may include the BDB in an amount of 10 to 40 μM and preferably 30 μM relative to the total weight of the nano-liposome in order to stabilize a dosage form.

Hereinafter, the present invention will be described in more detail with reference to Examples. However, those Examples are only for illustrating the present invention in more detail, but the range of the present invention is not intended to be limited to those Examples.

### EXAMPLE

Statistical Analysis

All measurements were performed in triplicate, and all values were expressed as the mean±the standard error. The results were subjected to an analysis of variance (ANOVA) using the Tukey's test to analyze differences between means. In each case, a P value of <0.05 was considered statistically significant.

Reagents

3-Bromo-4,5-dihydroxylbenzaldehyde (BDB, Matrix Scientific, Columbia, S.C., USA), N-acetyl cysteine (NAC), 5,5-dimethyl-1-pyrroline-N-oxide (DMPO), 2′,7′-dichlorodihydrofluorescein diacetate (DCF-DA), [3-(4,5-dimethylthiazol-2-yl)-2,5-diphenyltetrazolium] bromide (MTT) and Hoechst 33342 dye were purchased from Sigma Chemical Company (St. Louis, Mo., USA). All other chemicals and reagents were of analytical grade.

### Example 1

Cell Culture

Human keratinocytes (HaCaT cells) were obtained from the Amore Pacific Company (Gyeonggi-do, Republic of Korea). Cells were maintained at 37° C. in an incubator with a humidified atmosphere of 5% CO₂. Cells were cultured in Dulbecco's modified Eagle's medium containing 10% heat-inactivated fetal calf serum, streptomycin (100 μg/mL) and penicillin (100 unit/mL).

### Example 2

BDB Effectiveness on UVB-Induced Apoptosis

The effect of BDB on the viability of HaCaT cells was assessed as follows. Cells were seeded in a 96-well plate at a density of 1×10⁵ cells/mL. Sixteen hours after plating, BDB was added at a concentration of at 10, 20, 30, 40, and 50 μM. To evaluate the ability of BDB to protect keratinocytes against UVB-exposure, BDB was added at a concentration of 10, 20 and 30 μM, and cells were exposed to UVB radiation one hour later and incubated at 37° C. for 24 h. Fifty microliter of MTT stock solution (2 mg/mL) was added to each well to yield a total reaction volume of 200 μl. After incubating the cells for 4 h, the plate was centrifuged at 800×g for 5 min, and the supernatants were aspirated. The formazan crystals in each well were dissolved in dimethylsulfoxide (150 μl), and the absorbance at 540 nm was read on a scanning multi-well spectrophotometer.

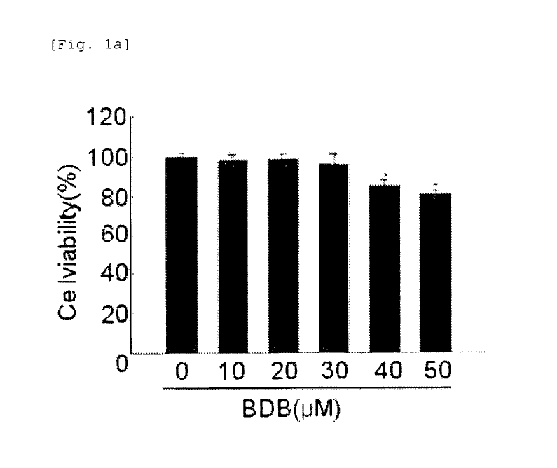

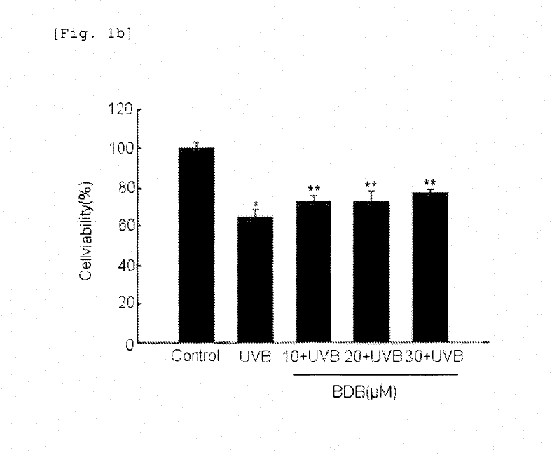

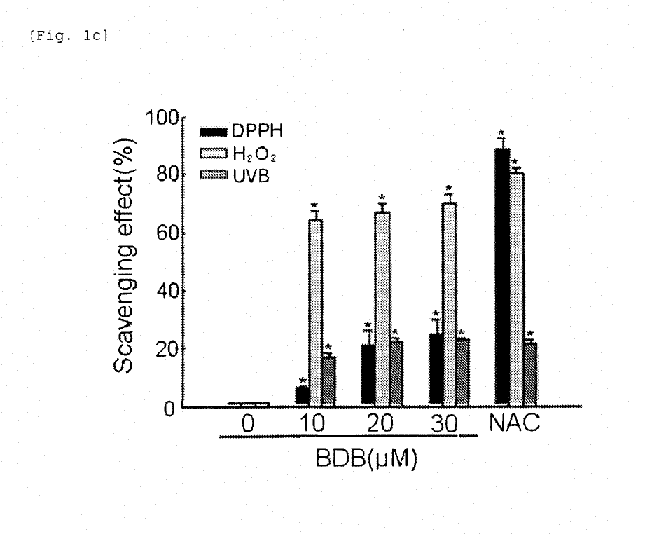

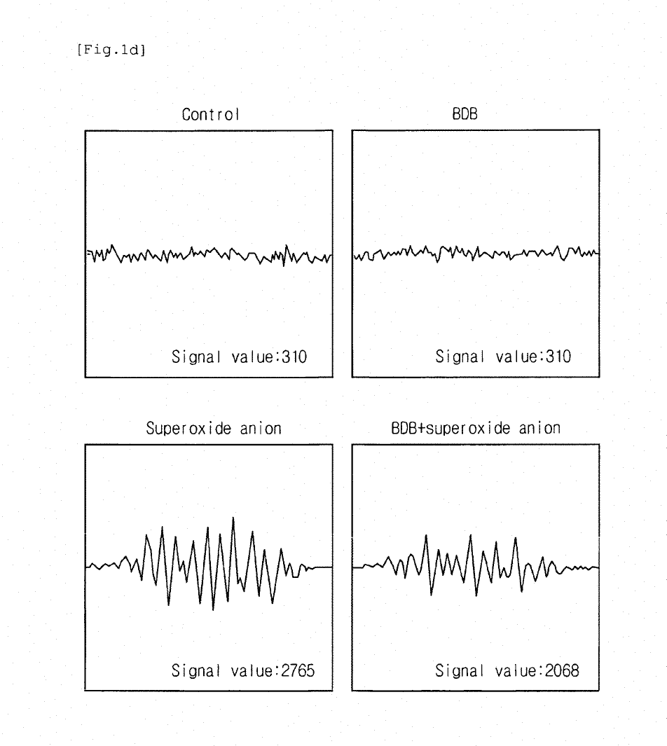

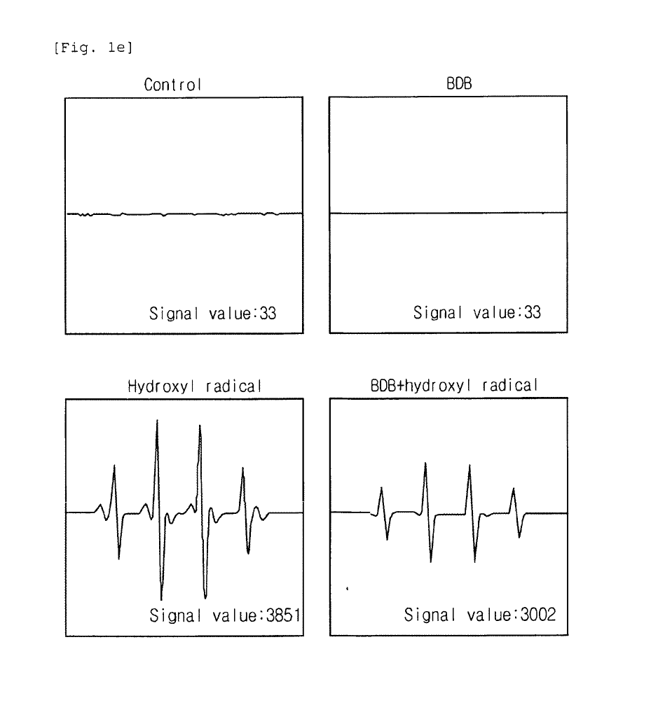

As a result, the BDB does not exhibit toxicity on human HaCaT keratinocyte up to the concentration of 30 μM (see FIG. 1a). As a measurement result of the viability of the cell exposed to UVB radiation, as illustrated in FIG. 1b, when the BDB's were treated in an amount of 10, 20, and 30 μM, the viabilities of the cell exposed to UVB were increased to be 73, 73, and 77%, respectively. Meanwhile, when the BDB was not treated, the viability of the cell exposed to UVB was 65%.

### Example 3

Free Radical Removal Ability of BDB

DPPH radical removal ability and intracellular reactive oxygen species (ROS) removal ability were measured in order to confirm free radical removal ability of the BDB of the present invention prepared from Example.

<3-1> Measurement of DPPH Radical Removal Ability

BDB at a concentration of 10, 20, 30 μM and 2 mM NAC were added to a 1×10⁻⁴ M solution of DPPH in methanol. The resulting reaction mixture was shaken vigorously. After 3 h, the amount of unreacted DPPH was measured at 520 nm using a spectrophotometer.

As a result, the BDB scavenged DPPH radical depending on the volume. As illustrated in a black bar in FIG. 1c, 10 μM of the BDB scavenged 6% of the DPPH radical, 20 μM of the BDB scavenged 21% of the DPPH radical, and 30 μM of the BDB scavenged 25% of the DPPH radical. Further, NAC (2 mM) that was known as a ROS scavenger and used as a positive control group scavenged 89%.

<3-2> Measurement of Intracellular Reactive Oxygen Species (ROS) Removal Ability

The DCF-DA method was used to detect intracellular ROS levels in HaCaT keratinocytes generated by either H₂O₂ or UVB radiation. For the detection of ROS in H₂O₂-treated cells, cells were seeded at a density of 1.5×10⁵ cells/well. Sixteen hours after plating, cells were treated with BDB at a concentration of 10, 20, 30 μM and 2 mM. NAC. After 30 min, H₂O₂ (1 mM) was added to the plate. Cells were incubated for an additional 30 min at 37° C., and DCF-DA solution (25 μM) was then added. Ten minutes after the addition of DCF-DA, the fluorescence of 2′,7′-dichlorofluorescein (DCF) was detected and quantified using a PerkinElmer LS-5B spectrofluorometer (PerkinElmer, Waltham, Mass., USA). For the detection of ROS in UVB-exposed cells, cells were treated with BDB as above. After one hour, cells were exposed to UVB radiation at a dose of 30 mJ/cm². The UVB source was a CL-1000M UV Crosslinker (UVP, Upland, Calif., USA), which was used to deliver an energy spectrum of rays (280 to 320 nm). Cells were incubated for an additional 24 h at 37° C., DCF-DA solution (25 μM) was added and detected as above.

As a result, it was confirmed that the BDB removes an intracellular ROS induced by H₂O₂. As illustrated with alight gray bar in FIG. 1c, 10 μM of the BDB removes 64%, 20 μM of the BDB removes 67%, and 30 μM of the BDB removes 70%, and also NAC removes 80%. Finally, as illustrated with a dark gray bar in FIG. 1c, 10 μM of the BDB removes 17% of an intracellular ROS induced by UVB, 20 μM of the BDB removes 22% of an intracellular ROS induced by UVB, and 30 μM of the BDB removes 23% of an intracellular ROS induced by UVB, and also NAC removes 22%. Based on the results as illustrated in FIG. 1, 30 μM concentration was determined as a proper concentration.

<3-3> Detection of Superoxide Anion

The superoxide anion was produced via the xanthine/xanthine oxidase system and then reacted with a nitrone spin trap, DMPO. The DMPO/.OOH adducts were detected using a JES-FA electron spin resonance (ESR) spectrometer (JEOL, Tokyo, Japan). Briefly, ESR signaling was recorded 5 min after 20 μl of xanthine oxidase (0.25 U/mL) was mixed with 20 μl each of xanthine (5 mM), DMPO (1.5 M) and BDB (30 μM). The ESR spectrometer parameters were set at a magnetic field of 336 mT, power of 1.00 mW, frequency of 9.4380 GHz, modulation amplitude of 0.2 mT, gain of 500, scan time of 0.5 min, scan width of 10 mT, time constant of 0.03 sec, and temperature of 25° C.

As the result of analyzing a removal effect of the BDB on the superoxide anion and hydroxyl radical by using an ESR spectrometry, as illustrated in FIG. 1d, the superoxide anion signal in the xanthine/xanthine oxidase system was increased by 2765 values, but when the BDB was treated, the superoxide anion signal was decreased to be 2063.

<3-4> Detection of Hydroxyl Radical

The hydroxyl radical was generated by the Fenton reaction (H₂O₂+FeSO₄) and then reacted with DMPO. The resultant DMPO/.OH adducts were detected using an ESR spectrometer. The ESR spectrum was recorded 2.5 min after a phosphate buffer solution (pH 7.4) was mixed with 0.2 mL each of 0.3 M DMPO, 10 mM FeSO₄, 10 mM H₂O₂, and BDB (30 μM). The ESR spectrometer parameters were set at a magnetic field of 336 mT, power of 1.00 mW, frequency of 9.4380 GHz, modulation amplitude of 0.2 mT, gain of 200, scan time of 0.5 min, scan width of 10 mT, time constant of 0.03 sec, and temperature of 25° C.

As a result, as illustrated in FIG. 1e, the hydroxyl radical signal in the Fenton reaction (H₂O₂+FeSO₄) system was increased by 3851, but was decreased to be 3002 by treating the BDB.

### Example 4

UVB Absorption Analysis

To study the UVB absorption spectra of BDB, which was diluted in DMSO at a ratio 1:500 (v/v), it was scanned by UV at 200-500 nm using Biochrom Libra S22 ultraviolet/visible spectrophotometer.

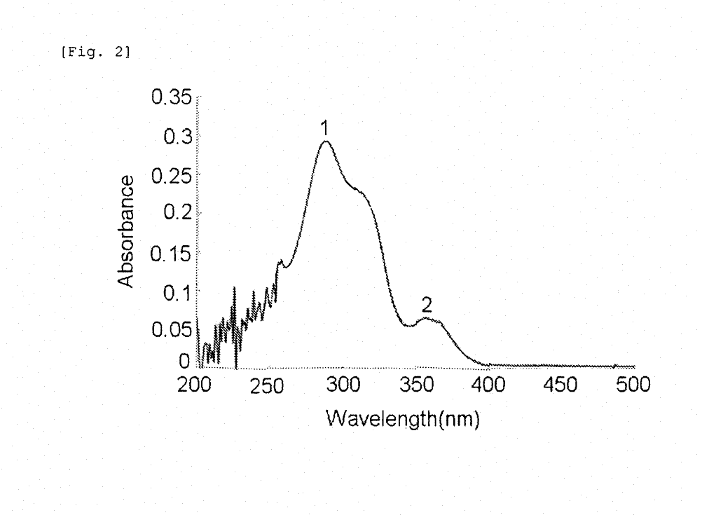

As a result, as illustrated in FIG. 2, the BDB exhibited the absorption ability at 280 to 320 nm that was a range of UVB. Accordingly, it was believed that the light absorption ability of the BDB related to a light protective effect in UVB radiation.

### Example 5

Lipid Peroxidation Inhibition Effect by BDB

A cell was exposed to UVB, and then after 24 hours, effects of BDB in inhibiting the cell membrane lipid peroxidation, a protein modification, and a cell DNA damage of UVB-irradiated cell was observed. Lipid peroxidation was assayed by the determination of 8-isoprostane levels in the culture medium. A commercial enzyme immunoassay (Cayman Chemical, Ann Arbor, Mich., USA) was employed according to the manufacturer's instructions. Lipid peroxidation was also estimated using a fluorescent probe, DPPP. Cells were incubated with 5 μM. DPPP for 15 min in the dark and then exposed to UVB. DPPP fluorescence image was captured using a Zeiss Axiovert 200 inverted microscope at an excitation wavelength of 351 nm and an emission wavelength of 380 nm and quantified.

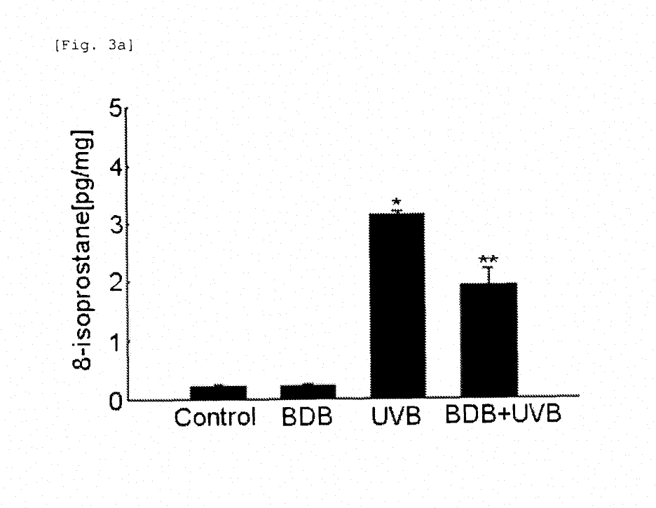

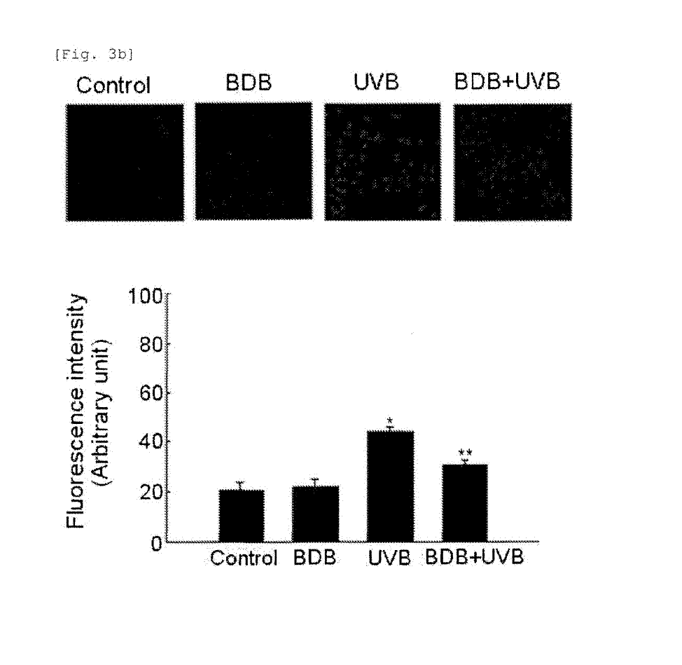

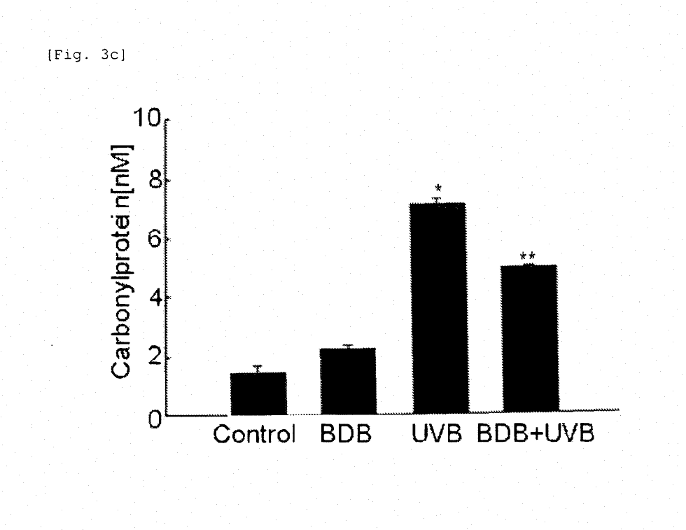

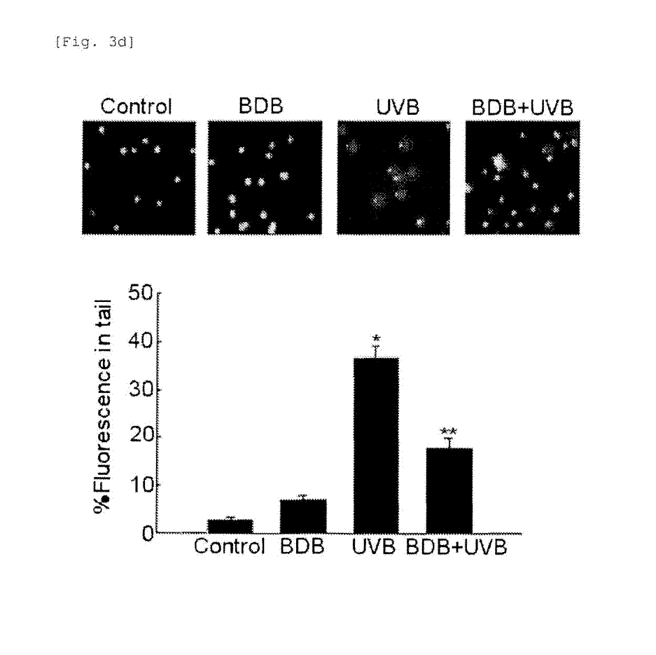

As a result, as illustrated in FIG. 3a, the UV-exposed cells had an increased 8-isopropane value (3.1 pg/mg). However, when the BDB was treated to the cell, a degree of increasing lipid peroxidation was inhibited (1.9 pg/mg). In addition, the lipid peroxidation was observed by using DPPP, in which the DPPP was reacted with the lipid hydroperoxide and produced highly fluorescent product DPPP oxide. The DPPP fluorescent strength was increased in the UVB-irradiated cell, but the BDB-treated cells exhibited the fluorescence in the more small range (see FIG. 3b).

### Example 6

Protein Carbonyl Formation Inhibition Effect by BDB

The amount of carbonyl formation in protein was determined using an Oxiselect™ protein carbonyl ELISA kit purchased from Cell Biolabs (San Diego, Calif., USA) according to the manufacturer's instructions.

As a result, as illustrated in FIG. 3c, the protein carbonyl level was increased in the UVB-irradiated cell, while the carbonyl formation induced by UVB was inhibited when treating with the BDB.

### Example 7

Protective Effect of BDB from DNA Damage

The degree of oxidative DNA damage was determined in a comet assay. Cell suspension was mixed with 75 μL of 0.5% low melting agarose (LMA) at 39° C. and the mixture was spread on a fully frosted microscopic slide pre-coated with 200 μL of 1% normal melting agarose (NMA). After solidification of the agarose, the slide was covered with another 75 μl of 0.5% LMA and then immersed in a lysis solution (2.5 M NaCl, 100 mM Na-EDTA, 10 mM Tris, 1% Trion X-100 and 10% DMSO, pH 10) for 1 h at 4° C. The slides were then placed in a gel-electrophoresis apparatus containing 300 mM NaOH and 10 mM Na-EDTA (pH 13) for 40 min to allow for DNA unwinding and the expression of the alkali-labile damage. An electrical field was then applied (300 mA, 25 V) for 20 min at 4° C. to draw the negatively charged DNA towards the anode. The slides were washed three times for 5 min at 4° C. in a neutralizing buffer (0.4 M Tris, pH 7.5), stained with 75 μL of propidium iodide (20 μg/mL) and observed using a fluorescence microscope and an image analyzer (Kinetic Imaging, Komet 5.5, UK). The percentage of total fluorescence in the DNA tails and the tail length of 50 cells per slide were recorded.

When a cell was exposed to UVB, the length and ratio of DNA tail to the cell tail were increased. When the cell was exposed to UVB, the ratio of DNA in the tail was increased to be 37%. In addition, as a result of treating with the BDB, as illustrated in FIG. 3d, it was decreased to be 18%. From such results, it can be confirmed that the BDB protects intracellular constitution components from oxidation damage caused by UVB.

### Example 8

Effect on Apoptosis Induced by UVB Irradiation

A direct relationship to apoptosis induced by UVB irradiation was investigated, and inhibition ability of BDB on apoptosis induced by UVB was investigated.

<8-1> Nuclear Staining with Hoechst 33342

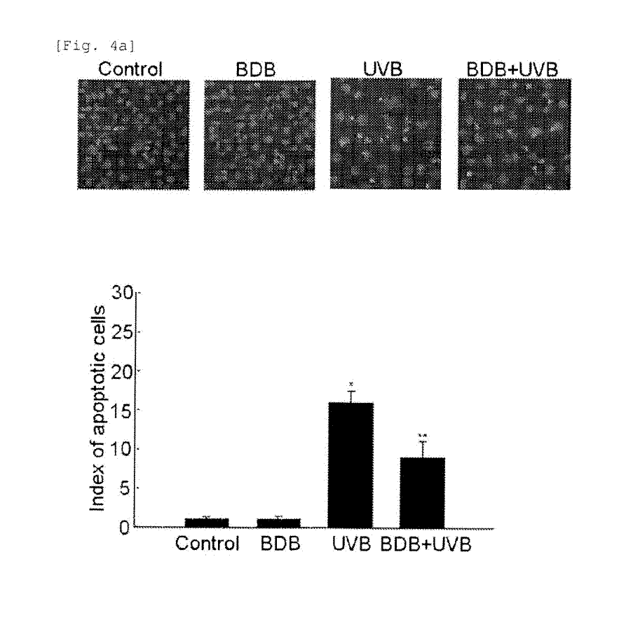

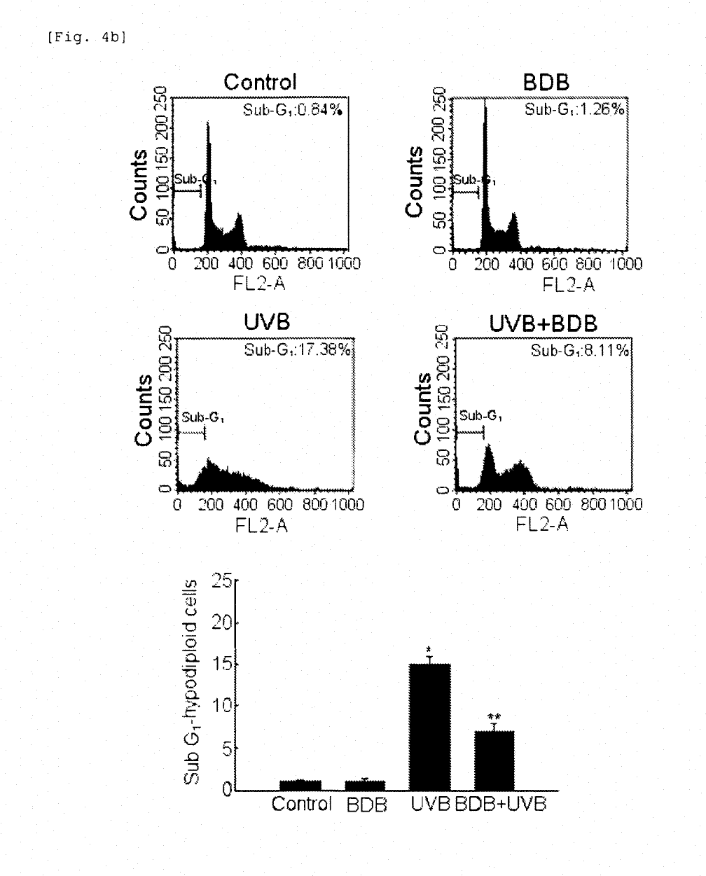

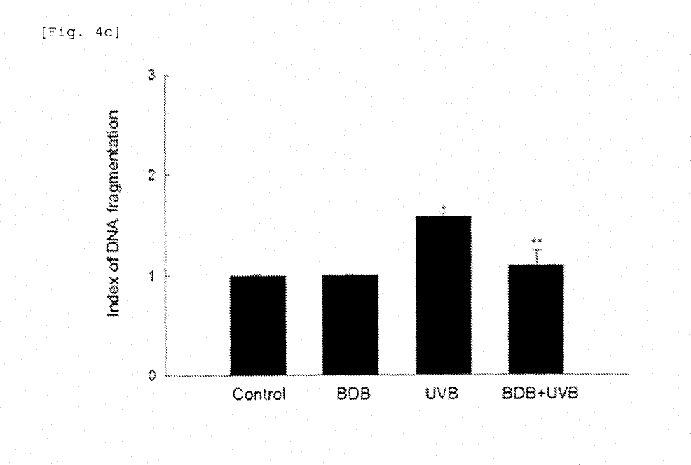

Cells were treated with BDB at a concentration of 30 μM and exposed to UVB radiation 1 h later. Cells were incubated for an additional 24 h at 37° C. Hoechst 33342 (1.5 μL of a 10 mg/mL stock), a DNA-specific fluorescent dye, was added to each well, and the cells were incubated for 10 min at 37° C. The stained cells were visualized under a fluorescence microscope equipped with a CoolSNAP-Pro color digital camera. The degree of nuclear condensation was evaluated, and the apoptotic cells were quantified. As a result, as illustrated in FIG. 4a, there were non-damaged cells in cells of a control group, but considerable nuclear segments were observed in the UVB-exposed cells (Apoptotic Index 16). However, the nuclear segments were significantly decreased in the UVB-irradiated cells treated with the BDB (Apoptotic Index 9).

<8-2> Sub-G₁ Hypodiploid Cells

Flow cytometry was performed in order to determine the apoptotic sub-G₁ hypodiploid cells. Cells were treated with BDB at a concentration of 30 μM and exposed to UVB radiation 1 h later. Cells were incubated for an additional 24 h at 37° C. Cells were harvested, and fixed in 1 mL of 70% ethanol for 30 min at 4° C. Cells were washed twice with PBS, and then incubated for 30 min in the dark at 37° C. in 1 mL of PBS containing 100 μg propidium iodide and 100 μg RNase A. Flow cytometric analysis was performed using a FACS Calibur flow cytometer (Becton Dickinson, Mountain View, Calif., USA). Sub-G₁ hypodiploid cells were assessed based on the histograms generated using the computer programs, Cell Quest and Mod-Fit.

In addition to a morphological evaluation, a protective effect of BDB on apoptosis can be confirmed by a flow cytometry. As a result of analyzing DNA in cells exposed to UVB, as illustrated in FIG. 4b, the apoptotic sub-G₁ DNA was increased to be 17%, while the cells without UVB exposure exhibited 1%. When the BDB was treated, the apoptotic sub-G₁ DNA was decreased to be 8%.

<8-3> DNA Fragmentation

Cellular DNA fragmentation was assessed by analyzing the extent of cytoplasmic histone-associated DNA fragmentation using a kit from Roche Diagnostics (Portland, Oreg., USA) according to the manufacturer's instructions. A cytoplasmic histone-associated DNA fragmentation was increased in UVB-irradiated cells as compared with control cells. The level of DNA fragmentation was decreased in UVB-irradiated cells that were treated with BDB (see FIG. 4c).

Accordingly, from the aforementioned results, the inventors of the present invention can found that the BDB effectively inhibits apoptosis confirmed by experiments of a degree of nuclear condensation and DNA fragmentation caused by ultraviolet irradiation; has ultraviolet absorption ability and free radical removal ability; inhibits a lipid peroxidation and protein carbonyl formation; and protects DNA damage; and thereby ultimately has excellent effect in protecting a cell from ultraviolet.

As set forth above, according to exemplary embodiments of the invention, the composition including a 3-bromo-4,5-dihydroxybenzaldehyde (BDB) has a photo-protective effect to cell damage induced by ultraviolet in human skin keratinocyte, and a free radical and reactive oxygen species-removal activities. Therefore, the BDB reduces apoptosis and recoveries cell viability, so that the BDB exhibits antioxidant activity, protects cell damage from ultraviolet, and also has an ultraviolet absorption effect. Thus, the BDB of the present invention can be usefully used as a raw material for a functional cosmetic composition or a pharmaceutical composition for protecting skin cell from ultraviolet.

While the present invention has been shown and described in connection with the exemplary embodiments, it will be apparent to those skilled in the art that modifications and variations can be made without departing from the spirit and scope of the invention as defined by the appended claims.

1. A method for inhibiting or treating skin damage caused by ultraviolet rays, comprising administering to the skin a composition comprising a 3-bromo-4,5-dihydroxybenzaldehyde (BDB) or salt thereof as an effective component.

2. (canceled)

3. The method according to claim 1, wherein the composition is in a type for external application of skin selected from the group consisting of cream, gel, a patch, a spraying agent, ointment, a hardening agent, lotions, liniments, pastes, and cataplasma.

4. The method according to claim 1, wherein the composition comprises the BDB in a concentration of 10 μM to 40 μM.

5-6. (canceled)

7. The method according to claim 1, wherein the composition is formulated into skin lotions, skin softeners, skin toners, astringents, lotions, milky lotions, moisture lotions, nutrition lotions, massage creams, nutrition creams, moisture creams, hand creams, essences, nutrition essences, packs, soaps, shampoos, cleansing foams, cleansing lotions, cleansing creams, body lotions, body cleansers, milky liquids, lipsticks, make-up bases, foundations, press powders, loose powders, or eye shadows.

8. (canceled)
